(19) United States
(12) Patent Application Publication
(10) Pub. No.: US 2026/0234356 A1
(43) Pub. Date: Aug. 13, 2026

(54) REDUCED CRYSTALLINITY POLYMERS FOR BIODEGRADATION

(71) Applicant: Battelle Memorial Institute, Columbus, OH (US)

(72) Inventors: Edward TRIGG, London, OH (US); Jacob Lilly, Dublin, OH (US); Amy Marie Heintz, Pacific Grove, CA (US); Gregory Gregoriades, Columbus, OH (US); Matthew Colachis, Columbus, OH (US); Ryan Daly, Columbus, OH (US)

(21) Appl. No.: 19/157,451

(22) PCT Filed: Feb. 28, 2024
(86) PCT No.: PCT/US24/17637

§ 371 Date: Aug. 18, 2025

Related U.S. Application Data
(60) Provisional application No. 63/487,688, filed on Mar. 1, 2023.

(51) Int. Cl. C08J 11/10

(52) U.S. Cl. CPC C08J 11/105; C08J 2367/02; C08J 2377/00

(57) ABSTRACT

The present invention is directed at the production of relatively reduced crystallinity and relatively high surface area polymer that is suitable for enzymatic degradation. In particular, the preparation of polyethylene terephthalate (PET) fibers from PET bottles with relatively reduced crystallinity and relatively higher surface area than the PET bottle feedstock. Such PET fibers are particularly suitable for enzymatic degradation into monomeric components.

### STATEMENT REGARDING FEDERALLY SPONSORED RESEARCH OR DEVELOPMENT

This invention was made with government support under DARPA, Grant No. TA3AOP, Contract No. G31290.XX.XX.0002.000001. The government has certain rights in the invention.

### FIELD

The present invention is directed at the production of relatively reduced crystallinity and relatively high surface area polymer that is suitable for enzymatic degradation. In particular, the preparation of polyethylene terephthalate (PET) fibers from PET bottles with relatively reduced crystallinity and relatively higher surface area than the PET bottle feedstock. Such PET fibers are particularly suitable for enzymatic degradation into monomeric components.

### BACKGROUND

Biodegradation and upcycling of recalcitrant plastics holds the promise of reducing waste and providing a cyclic source of chemicals while using little energy. Semicrystalline polymers are often processed to maximize crystallinity to improve properties. It is reported, however, that enzymes are relatively more efficient at attacking amorphous polymer than crystalline polymer. See, e.g., Thomsen et al., New Biotechnology, 2022, 69, 28-35.

### SUMMARY

A method for enzymatic degradation of a semicrystalline polymer comprising: (1) supplying a semicrystalline polymer susceptible to enzymatic degradation having a first crystallinity C₁ and a first surface area SA₁; (2) processing said semicrystalline polymer to provide a second crystallinity C₂ and a second surface area SA₂, wherein C₂<C₁ and SA₂>SA₁; (3) subjecting said semicrystalline polymer provided in step (2) to enzymatic degradation.

A method for enzymatic degradation of a semicrystalline polymer comprising: (1) supplying a semicrystalline polyester susceptible to enzymatic degradation having a first crystallinity C₁ of 15.0 to 50.0% and a first surface area SA₁; (2) processing said semicrystalline polyester into filaments having a diameter in the range of 10 μm to 200 μm and cooling at a rate of at least 0.5° C./ms to provide a second crystallinity C₂ in the range of 5.0% to 15.0% and a second surface area SA₂, wherein C₂<C₁ and SA₂>SA₁; (3) subjecting said semicrystalline polyester filaments provided in step (2) to enzymatic degradation.

### FIGURES

FIG. 1 illustrates the effect of melt spinning with a reduction in crystallinity and increase in surface area of PET bottle feedstock that is shredded, melted and melt spun and cooled into filaments (extruded fiber).

FIG. 2 illustrates the enzymatic degradation, as measured by the observed level of terephthalic acid (TA) produced, from an original PET bottle, extruded PET fibers, and a PET film control.

FIG. 3 illustrates process of an exemplary PET bottle via shredding, extrusion to form filaments and spooling of the filaments.

FIG. 4 illustrates another exemplary procedure herein for the formation of semicrystalline polymer filaments herein with reduced crystallinity and relatively higher surface area than the bottle feedstock.

FIG. 5 illustrates a rotating spool for collection of the polymer filaments herein with reduced crystallinity and relatively higher surface area than the bottle feedstock.

FIG. 6 illustrates the surface area/volume (SA/V) of a sample PET cup, shredded PET cup, and a spooled/extruded cup.

FIG. 7 is a plot of SA/V versus spooler speed at a preferred 18″ drop distance.

### DETAILED DESCRIPTION OF PREFERRED EMBODIMENTS

The present invention relates to a method for production of reduced crystallinity fibers with relatively high surface areas that are suitable for enzymatic degradation. Initially, a feedstock of one or more semicrystalline polymers is identified that are susceptible to enzymatic degradation, where such semicrystalline polymers preferably include polymers such as polyethylene terephthalate (PET) and/or aliphatic polyamides (e.g., nylon-6 or nylon-6,6). Accordingly, the semicrystalline polymers herein suitable for a biodegradation (e.g., enzymatic degradation) protocol include those that preferably contain ester or amide bonds. Reference to semicrystalline polymer is reference to the feature that the polymer contains some level of both crystalline and amorphous domains.

The semicrystalline polymers therefore have an initial or first level of crystallinity (C₁) and surface area (SA₁). The surface area may be measured in m²/gram. Upon processing herein, the semicrystalline polymers are converted into a second physical form where the level of crystallinity (C₂) is such that C₂<C₁. In addition, upon such processing the semicrystalline polymers provide a second surface area (SA₂) where SA₂>SA₁. The aforementioned first level of crystallinity (C₁) may preferably fall in the range of greater than 15.0% to 50.0%.

Preferably, the processing relied upon herein is melt spinning, which is a general reference to the feature of melting and extruding the semicrystalline polymer and passing the molten polymer through a spinneret and forming filaments followed by spooling. However, when forming the filaments herein, the conditions for spooling are now preferably adjusted to provide a cooling profile so that the filaments form with relatively reduced crystallinity (C₂) and relatively increased surface area (SA₂) as compared to the feedstock.

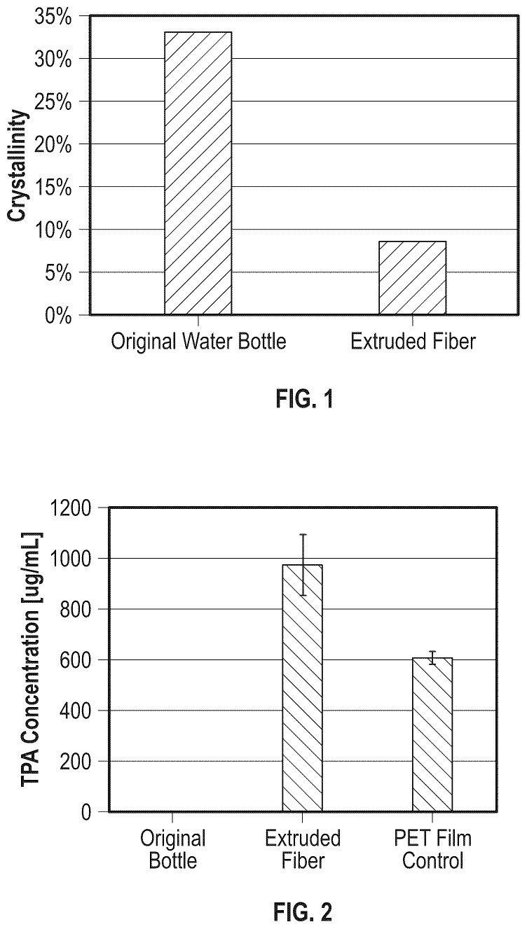

Attention is directed to FIG. 1, which is a working example of the preferred approach herein as applied to melt spinning with reduction in crystallinity and increase surface area of a PET bottle feedstock. As can be seen in FIG. 1, the PET water bottle feedstock indicated a first crystallinity (C₁) of about 33%. The PET water bottle feedstock may then be preferably shredded, melted and melt spun and cooled into filaments (identified in FIG. 1 as “Extruded fiber”). The rate of spooling and rate of cooling are adjusted to provide relatively rapid cooling and a reduced second crystallinity (C₂), as shown in FIG. 1, of about 9.0%. However, in the broad context of the present invention, such second crystallinity (C₂) may fall in the range of 5.0% to 15.0%, including all individual values and increments therein. Therefore, the second crystallinity may be 5.0%, 6.0%, 7.0%, 8.0%, 9.0%, 10.0%, 11.0%, 12.0%, 13.0%, 14.0% or 15.0%. In addition, such crystallinity is conveniently measured by differential scanning calorimetry (DSC) testing. Moreover, the cooled filaments so formed from melt spinning are contemplated to have a preferred diameter in the range of 10 μm to 200 μm, including all individual values and increments therein. More preferably the cooled filaments therefore have a diameter in the range of 10 μm to 100 μm.

It is worth mentioning that in connection with the above-described procedure, typically, PET bottles are shredded and extruded and formed into pellets, which are then melt spun. In the present invention, the shredded bottles can be converted directly into the filaments having the reduced crystallinity and increased surface area without a need to initially convert the shredded bottles into pellets. Moreover, as noted, the filament diameter may fall in the range of 10 μm to 200 μm, and the filament diameter need not be regulated so that it is uniform. Accordingly, the filament diameter may vary within the identified preferred range of 10 μm to 200 μm.

Attention is next directed to FIG. 2 which illustrates the enzymatic degradation rate for the cooled filaments (“Extruded fiber”) produced in FIG. 1. More specifically, upon treatment with a variant of the leaf compost-cutinase enzyme (LCC-ICCG), for 24 hours at 60° C., at an enzyme to polymer ratio of 1 mg/g, the ensuing level of terephthalic acid (TPA) was measured. The TPA concentration can be considered as proportional to the depolymerization rate of PET. As can be observed, enzymatic treatment of the original feedstock bottle effectively offered no depolymerization, and the depolymerization of the melt spun fibers into the cooled filaments (“Extruded fiber”) outperformed in enzymatic degradation by providing a relatively higher TPA concentration than a PET film control. The PET film control shown in FIG. 2 has a crystallinity of about 6.7%+/−2.1%. However, such film control has a relatively lower surface/volume than the extruded fiber.

Elaborating on the above, it can be appreciated that in the case of semi-crystalline polymers targeted for enzymatic degradation, the crystallinity of such polymers may now be more conveniently reduced or eliminated during fiber formation, by relatively rapidly cooling from the molten state to a temperature below the glass transition temperature (Tg). The enzyme that may be employed are preferably those that promote, e.g., breakdown of the covalent bonds present in a given semicrystalline polymer. For example, breakdown of ester bonds or amide bonds in a given polymer chain. Non-limiting and preferred examples of enzymes therefore include: cutinase, lipase, esterase, carboxylesterase, p-nitrobenzylesterase, protease, serine protease, amidase, aryl-acylamidase, urethanase, oligomer hydrolase, laccase, peroxidase, haloperoxidase, lipoxygenase, mono-oxygenase, di-oxygenase and hydroxilase.

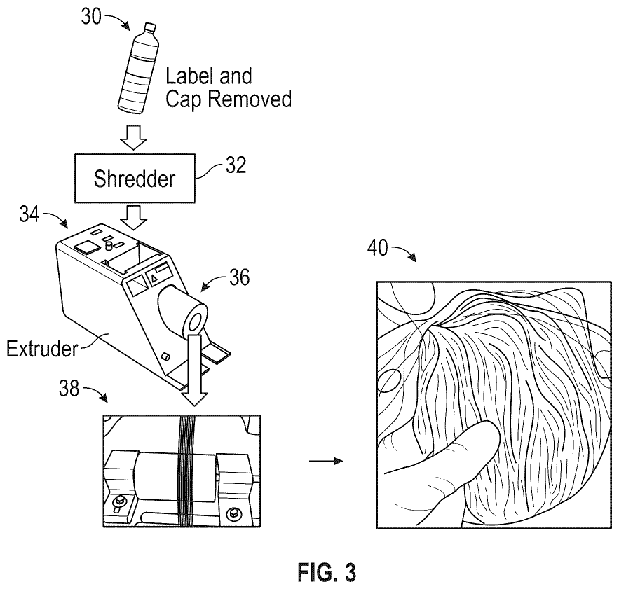

Attention is directed to FIG. 3, which illustrates that the PET bottle 30 is first processed to remove the label and cap and then preferably shred at 32 to a particle size in the preferred range of 1 mm to 5.0 mm. The shredded PET bottle is then fed into an extruder 34 and passed through a spinneret 36 to form filament(s) which are then passed to a spooler 38 which leads to the ability to collect filament(s) 40. As also seen in FIG. 3, the output filaments of the extruder spinneret 36 preferably undergo what may be termed a drop at arrow 39. The drop distance is therefore the distance between the extruder spinneret nozzle and the top of the spool. The distance of this drop may preferably range from 6 inches to 60 inches, more preferably from 10 inches to 20 inches, and most preferably, 15 inches to 20 inches, including all individual values and ranges in such aforementioned ranges. By way of one particularly preferred example, the drop may therefore be 15 inches, 16 inches, 17 inches, 18 inches, 19 inches or 20 inches.

Extruder screw speed is preferably at or above 13 rpm, and in the range of 13 rpm to 60 pm. More preferably, the extruder screw speed is in the range of 30 rpm to 60 rpm, including all individual values and increments therein. Accordingly, a more particularly preferred extruder screw speed is in the range of 40 rpm to 50 rpm. The temperature of the PET in the extruder is preferably in the range of 260° C. to 290° C. It is also preferred to include an impeller (agitator) in the feed hopper of the extruder to agitate the shredded PET and reduce or eliminate static and reduce or eliminate bridging in the extruder feed throat.

The cooling on the spooler 38 may be preferably achieved by extruding the molten polymer filament(s) onto the spool 38 that is preferably rotating in the range of 100 rpm to 2000 rpm, including all individual values and increments therein and where the top surface of the spooler 38 is as noted is preferably positioned 6 inches to 60 inches from the spinneret orifice 36. The filament(s) then undergo a relatively rapid cooling in ambient air, particularly as the filament(s) as noted may have a preferred diameter in the range of 10 μm to 200 μm. Accordingly, the spooler itself preferably does not rely upon internal refrigeration, i.e. it preferably is not chilled. As a consequence of such relatively rapid cooling, the crystallinity of the semicrystalline PET in the PET bottle at 30 is reduced and the surface area is increased for ensuing enzymatic degradation. The cooling rate of the molten polymer filaments on the spool is preferably at least 0.5° C./ms, and in the range of 0.5° C./ms to 5.0° C./ms including all individual values and increments therein. For example, another preferable range is 1.0° C./ms to 2.0° C./ms.

It is noted that the above referenced shredding, extrusion and cooling on the spooler is now configured such that the power consumption for a batch of 500 grams of PET will fall in the range of 0.50 kWh to 1.00 kWh, more preferably in the range of 0.80 kwH to 0.90 kWh.

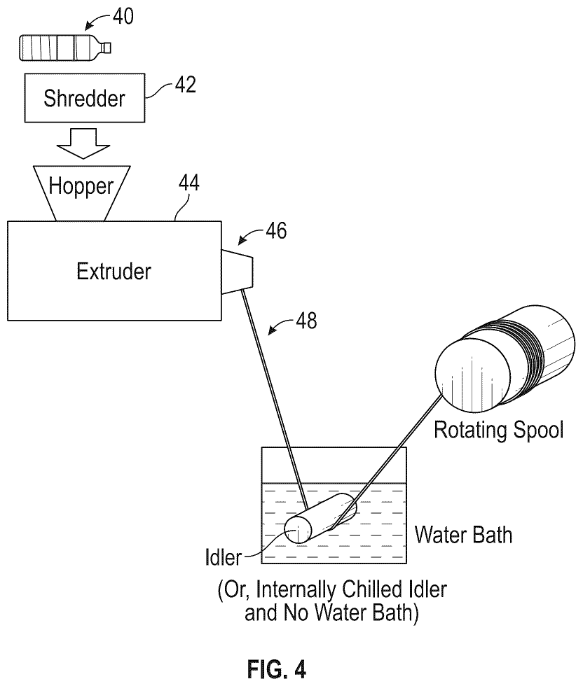

FIG. 4 illustrates another procedure for formation of the semicrystalline polymer herein with relatively reduced crystallinity and relatively higher surface area, from the semicrystalline polymer feedstock. Once again, one preferably employs a PET bottle 40 that is processed to remove the label and cap and then again shred at 42 to a particle size in the range of 1 mm to 5.0 mm. The shredded PET bottle is then fed into an extruder 44 and through a single orifice spinneret 46. One may also use a multi-orifice spinneret as noted above. The filament(s) 48 may then be submerged in a water bath 48 which can provide relatively rapid cooling to reduce or eliminate polymer crystallization. Such cooling rate is again contemplated to be in the range of 0.5° C./ms to 5.0° C./ms including all individual values and increments therein. It is also contemplated herein that in lieu of a water bath, one may utilize a blower assembly to force air, with or without cooling, directly on the filament(s) to again reduce or eliminate the relative amount of crystalline polymer that would otherwise form.

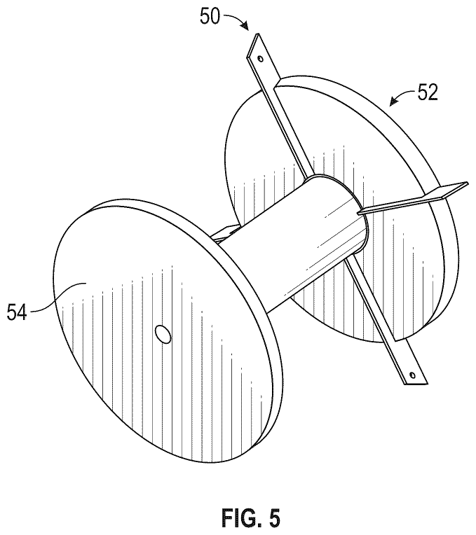

The filament(s) formed in FIG. 3 or FIG. 4 herein, having a relatively reduced level of crystallinity and relatively higher surface area than the PET bottle feedstock, may be conveniently transferred to an enzymatic biodegradation reactor. It is also contemplated that such filaments may be cut with a blade or scissors. In addition, as illustrated in FIG. 5, the rotating spool for collection of the filaments may be configured with blades 50 incorporated into the spool. The flange 52 containing the blades may then be pressed against the opposing flange 54 to execute the cut of the filaments.

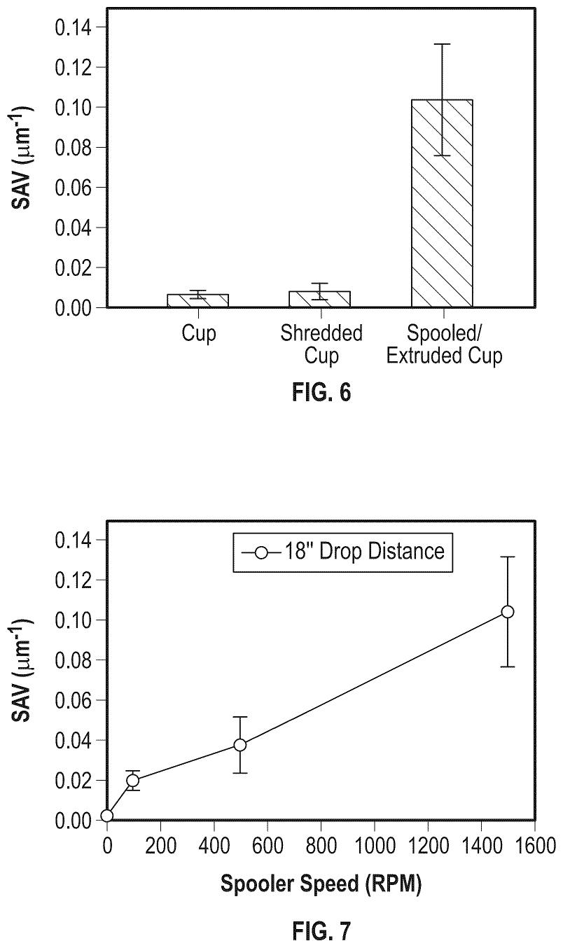

Attention is next directed to FIGS. 6 and 7. FIG. 6 illustrates the surface area/volume (SA/V) of a sample PET cup, shredded PET cup, and a spooled/extruded cup. The spooled/extruded cup therefore is reference to the PET filaments produced herein with the second crystallinity (C₂) and second surface (SA₂) area where the SA/V of such filaments preferably fall in the range of 0.07 μm⁻¹ to 0.14 μm⁻¹. FIG. 7 next is a plot of SA/V versus spooler speed at a preferred 18″ drop distance for the spooled/extruded cup of FIG. 6. As can be observed, increasing spooler speed increases the SA/V of the PET filaments that are produced herein.

It may therefore be appreciated that the present invention stands directed at a method for enzymatic degradation of a semi-crystalline polymer comprising: (1) supplying a semicrystalline polymer susceptible to enzymatic degradation having a first crystallinity C₁ and a first surface area SA₁; (2) processing said semicrystalline polymer to provide a second crystallinity C₂ and a second surface area SA₂, wherein C₂<C₁ and SA₂>SA₁; (3) subjecting said semicrystalline polymer provided in step (2) to enzymatic degradation. The method is particular applicable to polyethylene terephthalate (PET) bottles and films that are targeted for recycling.

Crystallinity Measurements: Preferably, crystallinity herein can be evaluated on a TA Instruments DSC25P high-pressure Differential Scanning calorimeter (DSC). One to two milligrams of material are sealed in a standard Tzero aluminum DSC pan, the cell was flushed with industrial nitrogen, and the test run at 10° C. per minute from 20° C. to 300° C. The crystallinity was measured using TA Instruments Trios Software. Crystallinity was calculated suing the following equation

$$\% Crystallinity = \frac{( Δ {H}_{m} - Δ {H}_{c} )}{Δ {H}_{m}^{∘}} \times 100 \%$$

where ΔHₘ was the normalized enthalpy of the melting endothermic peak, ΔH_{c} was the normalized enthalpy of the cold crystallization exothermic peak, and ΔH^{o}ₘ was the theoretical endothermic heat of melting of 100% crystalline PET. In addition, ΔHₘ and ΔH_{c} was attributed to the area under the endotherm and exotherm DSC curves, respectively.

Fiber Diameter: The diameter of the fibers evaluated herein was performed using a KeyenceVK-X260K Optical Laser Scanning Confocal microscope at 10× magnification. Data was analyzed using the VK-H2X observation/analysis software. Five diameter measurements were recorded per fiber image at three different regions along each fiber sample. The average fiber diameter was calculated for each sample.

Surface Area: The surface area of the cup was approximated using equation (1) below, where r₁ was the radius of the rim, r₂ was the radius of the cup bottom, and h was the height of the cup. The volume of the cup was approximated using equation (2) below, where r₁ was the radius of the rim, r₂ was the radius of the cup bottom, l₁ was the average measured thickness of the cup wall, and l₂ was the average measured thickness of the cup bottom. The rim of the cup was not included in the calculations as the rim was not included in the shredded pieces that were processed.

$$( 1 ) SA ( cup ) = 2 ( π {r}_{1} + π {r}_{2} ) \sqrt{{( {r}_{1} - {r}_{2} )}^{2} + {h}^{2}} + 2 π {r}_{2}^{2}$$

$$( 2 ) Volume ( cup ) = {l}_{1} ( π {r}_{1} + π {r}_{2} ) \sqrt{{( {r}_{1} - {r}_{2} )}^{2} + {h}^{2}} + {l}_{2} π {r}_{2}^{2}$$

The average SA/V of the cup throughout measured portions is then calculated by the quotient of the surface area divided by the volume.

The shredded PET SA/V was calculated using average dimensions taken from samples of shredded PET. These calculations were done under the assumption that all PET shreds were rectangular. Thus, equation 3 gives the equation for surface area of a l by w rectangular prism with thickness t, and equation 4 gives the equation for volume of that geometry. Equation 5 combines these equations to give the equation for SA/V of a rectangular shredded flake.

$$( 3 ) SA ( shred ) = 2 ( lw + lt + wt )$$

$$( 4 ) Vol ( shred ) = lwt$$

$$( 5 ) \frac{SA}{V} ( shred ) = \frac{2}{l} + \frac{2}{w} + \frac{2}{t}$$

Finally, the SA/V was estimated for the extruded fiber, using equations 6, 7, and 8 for surface area, volume, and SA/V, respectively. These equations approximate the geometry of the fiber as an infinitely long cylinder, as the surface area of the circular faces are negligible. Thus, equation 8 demonstrates a derived formula for the SA/V of the extruded fiber as a function of the fiber diameter only. In the equations, d is the fiber diameter and l is the fiber length.

$$( 6 ) SA ( fiber ) = π dl$$

$$( 7 ) Vol ( fiber ) = π {l ( \frac{d}{2} )}^{2}$$

$$( 8 ) \frac{SA}{V} ( fiber ) = \frac{π dl}{π {l ( \frac{d}{2} )}^{2}} = \frac{4}{d}$$

1. A method for enzymatic degradation of a semicrystalline polymer comprising: (1) supplying a semicrystalline polymer susceptible to enzymatic degradation having a first crystallinity C₁ and a first surface area SA₁; (2) processing said semicrystalline polymer to provide a second crystallinity C₂ and a second surface area SA₂, wherein C₂<C₁ and SA₂>SA₁; (3) subjecting said semicrystalline polymer provided in step (2) to enzymatic degradation.

2. The method of claim 1 wherein said semicrystalline polymer susceptible to enzymatic degradation comprises a polymer containing ester bonds.

3. The method of claim 2 wherein said polymer containing ester bonds comprises polyethylene terephthalate (PET).

4. The method of claim 1 wherein said semicrystalline polymer susceptible to enzymatic degradation comprises a polymer containing amide bonds.

5. The method of claim 1 wherein said semicrystalline polymer susceptible to enzymatic degradation comprises a PET bottle.

6. The method of claim 1 wherein step (2) comprises melting and extruding said semicrystalline polymer and passing the molten polymer through a spinneret and forming filament(s).

7. The method of claim 1 wherein said first crystallinity (C₁) of said semicrystalline polymer is in the range of greater than 15.0% to 50.0%.

8. The method of claim 1 wherein said second crystallinity (C₂) of said processed semicrystalline polymer is 5.0% to 15.0%.

9. The method of claim 1 wherein processing said semicrystalline polymer to provide a second crystallinity C₂ and a second surface area SA₂ comprises cooling at a rate of at least 0.5° C./ms

10. The method of claim 9 wherein said cooling rate is in the range of 0.5° C./ms to 5.0° C./ms.

11. The method of claim 1 wherein the semicrystalline polymer provided in step (2) has a surface area/volume (SA/V) value in the range of 0.07 μm⁻¹ to 0.14 μm⁻¹.

12. A method for enzymatic degradation of a semicrystalline polymer comprising: (1) supplying a semicrystalline polyester susceptible to enzymatic degradation having a first crystallinity C₁ of 15.0 to 50.0% and a first surface area SA₁; (2) processing said semicrystalline polyester into filaments having a diameter in the range of 10 μm to 200 μm and cooling at a rate of at least 0.5° C./ms to provide a second crystallinity C₂ in the range of 5.0% to 15.0% and a second surface area SA₂, wherein C₂<C₁ and SA₂>SA₁; (3) subjecting said semicrystalline polyester filaments provided in step (2) to enzymatic degradation.

13. The method of claim 12 comprising processing said semicrystalline polyester into filaments having a diameter in the range of 10 μm to 100 μm.

14. The method of claim 12 wherein said cooling is at a rate of 0.5° C./ms to 5.0° C./ms.

15. The method of claim 12 wherein said cooling is at a rate of 1.0° C./ms to 2.0° C./ms.

16. The method of claim 12 wherein said semicrystalline polyester provided in step (2) has a surface area/volume (SA/V) value in the range of 0.07 μm⁻¹ to 0.14 μm⁻¹.

17. The method of claim 12 wherein said semicrystalline polyester comprises polyethylene terephthalate (PET).
